(19) United States
(12) Patent Application Publication
(10) Pub. No.: US 2007/0162096 A1
(43) Pub. Date: Jul. 12, 2007

(54) Temperature regulating suit

(75) Inventors: Mordechai Zakuto, Bat-Yam (IL); Arik Mendyuk, Neve Savyon (IL); Dror Brotfeld, Hod-HaSharon (IL)

Correspondence Address: Martin D. Moynihan;PRTSI, Inc., P.O. Box 16446, Arlington, VA, 22215, US

(73) Assignee: Medisuit Ltd., Tel-Aviv (IL)

(21) Appl. No.: 11/329,085

(22) Filed: Jan. 11, 2006

(51) Int. Cl. A61F 7/00

(52) U.S. Cl. 607/104; 607/108; 607/114

(57) ABSTRACT

A device for the controlled heating or cooling of a portion of the body is disclosed. The device includes a contact surface; a plastically deformable, substantially sheet element configured to define the shape of the contact surface; and a heat transfer component, associated with the plastically deformable element, for adjusting the temperature of the contact surface.

### FIELD AND BACKGROUND OF THE INVENTION

The present invention relates to the field of temperature regulation of a body and, more particularly, to a method and a device useful for the controlled heating or cooling of a person, for example, of a patient during surgery.

Generally, it is important to be able to control the temperature of a body of a person. Thus, controlled heating or cooling of a patient may be required under various circumstances.

Cooling of a patient is important in various situations. For example, in cases where a patient has experienced cardiac arrest, a stroke, or traumatic brain injury, cooling provides neuroprotection, improving patient outcome. Cooling may also be required for maintaining the body temperature of a heat sensitive or feverish patient. Local cooling of a portion of the body of a patient, such as a specific area or a limb, may be required in order to reduce possibly dangerous swelling such as during the first 24 hours of a closed compartment wound.

There is a need for a device and a method for the controlled cooling of at least a portion of the body of a person.

Heating of a patient is important in various situations such as during transport in an ambulance, for example, of a person having hypothermia or in cases of accident victims who are partially or totally unclothed due to rescue efforts. Other situations where the heating of a patient is required include, for example, in cases of patients who are anesthetized for surgery and consequently are unable to maintain an own body temperature. Heating of such patients prevents their body temperatures from dropping below a desired temperature and reduces bodily stress resulting from surgery. Frail or elderly patients may require heating. Sometimes, local heating of portion of the body of a patient, such as a specific area or a limb, may be required in order to increase blood flow to rid the body of toxins, such as in the case of a closed compartment wound subsequent to the first 24 hours.

A factor which must be considered when a patient is to undergo surgery or other medical procedure is the necessity of providing a clean and safe operating environment for the patient. While the contents of an operating room may be sterilized prior to use, operating room personnel may inadvertently introduce bacteria or other microorganisms into the operating room. In order to ensure that the surgical environment is maintained as sterile as possible, it is common practice to lower the temperature of the operating room, reducing the rate of development of such microorganisms. When the temperature of an operating room is reduced, as discussed above, it is necessary to heat a patient, so as to maintain his temperature at a constant level. Heating of a patient may be facilitated by means of various known devices, such as a combination of blankets and heating elements applied to the skin of the patient or heating elements associated with the surface on which the patient is resting. Known heating devices are disadvantageous, in not providing even heating of a patient, are difficult to control especially during a long surgical procedure, and pose the risk of causing burn wounds to the patient.

There is a need for a device and a method for the controlled heating of at least a portion of the body of a person.

In the art, various methods and devices for the heating or cooling of a patient are known.

U.S. Pat. No. 6,473,910 discloses a multi-layered garment for cooling the upper portion of the body. The garment includes an outer layer which is liquid impermeable and vapor permeable, a central absorbent layer, and an inner layer which is liquid permeable. In use, the garment is saturated with a cool liquid and is worn on the upper body, whereby the effect of evaporative cooling provides the wearer with relief from heat. The garment is, however, neither suitable for heating a patient nor suitable for use on a patient during surgery.

MTRE Advanced Technologies, Ltd., of Or Akiva, Israel, has developed a system for thermoregulation, whereby a one-piece garment is wrapped around a patient. The garment is formed of two exterior layers of a non-woven fabric and two inner layers of non-PVC plastic film. Water at a predetermined temperature is caused to flow through the volume defined by the two inner layers. The garment is held in place by adhesive strips and, if necessary during treatment of the patient, portions of the garment may be opened and later retaped as necessary. Although the one-piece garment is intended to be suitable for all patients, and ten different sizes of the one-piece garment are available for adults, children, and infants, the garments are ill fitting to most patients. Further, due to the particular construction of the garment, it is unsuitable for use with a patient who is injured, as portions of the body of the patient must be moved in order to properly position the garment and to secure its adhesive strips. Additionally, portions of the garment do not overlap and portions of the patient's body may not be wrapped at all, such that there is incomplete coverage of the patient's body, resulting in inefficient heating. Another distinct disadvantage of the garment is that it is provided with a single water inlet and a single water outlet such that, when heated water is pumped through the garment, the portion of the body near the inlet may be overheated or even burned, while the portion of the body near the outlet may not be heated sufficiently. Also, due to the construction of the garment and its positioning around the body, water pressure on the body, at certain points inside the garment, may be so great as to restrict circulation or to even cause damage to the body. Additionally, when a patient has been wrapped in the garment, local regions of his body may not be easily accessible, such as may be required to administer an injection or to perform a medical procedure such as, for example, an appendectomy. Furthermore, the garment is very complex to manufacture and to sterilize and, as a result, is very expensive to produce.

It would be highly advantageous to have means for the controlled heating or cooling of a patient, which would overcome at least some of the disadvantages of the prior art. Preferably, the device should be suitable for heating or cooling of at least a portion of the body of a patient. It should be safe to use, such that there is no danger of burning the patient. It should be simple to use, sterile, and inexpensive to produce. While the device should be suitable for all adults, children, and infants, there should not be required more than, at most, a few sizes of the device.

### SUMMARY OF THE INVENTION

The present invention successfully addresses at least some of the disadvantages of the prior art by providing a device for the controlled heating or cooling of a portion of the body. The device comprises a plastically deformable, substantially sheet element configured to define the shape of a contact surface and a heat transfer component, associated with the plastically deformable element, for adjusting the temperature of the contact surface. According to an embodiment of the invention, the heat transfer component comprises first and second deformable waterproof layers which define a fluid chamber having an inlet and an outlet, the cross-sectional area of the fluid chamber having a size and configuration corresponding substantially to those of the plastically deformable element; the plastically deformable element being disposed between the first and second waterproof layers.

According to the teachings of the present invention there is provided a device for the controlled heating or cooling of a portion of the body, comprising: a contact surface; a plastically deformable, substantially sheet element configured to define the shape of the contact surface; and a heat transfer component, associated with the plastically deformable element, for adjusting the temperature of the contact surface. In embodiments of the present invention, the contact surface is configured to make contact with a portion of the body such that either heat is transferred from the body portion to the contact surface or heat from the body is transferred to the contact surface.

In embodiments, the heat transfer component comprises a chamber having walls of a flexible material. In embodiments, the chamber is functionally associated with the deformable sheet element such that the deformable sheet element substantially defines the shape of the chamber. In embodiments, the deformable sheet element is found substantially within the chamber. In embodiments, the deformable sheet element is found substantially outside the chamber. In embodiments, the walls of the chamber are substantially waterproof. In embodiments, the size and shape of the chamber are substantially similar to the size and shape of the plastically deformable element.

According to the teachings of the present invention the flexible material is elastic. In embodiments, the walls of the chamber define the contact surface. In embodiments, the device further comprises an external layer defining the contact surface, the external layer in intimate contact with at least one of the walls of the chamber. In embodiments, the external layer is a padding layer, a compressible layer, an elastically compressible layer, and/or an absorbent layer. In embodiments, the external layer is any material selected from the group consisting of non-woven fabric, woven fabric, fiber fabric, mesh fabric, knitted fabric, cloth, cotton, terry cloth, foam, and polyurethane. In embodiments, the external layer is in direct intimate contact with the at least one wall.

In embodiments, between the external layer and the at least one wall there is at least one further layer, so that the external layer and the at least one wall are in indirect intimate contact. In embodiments, the at least one further layer is an adhesive layer, a padding layer, a compressible layer, an elastically compressible layer, and/or an absorbent layer. Suitable materials from which to make such a further layer include but are not limited to non-woven fabrics, woven fabrics, fiber fabrics, mesh fabrics, knitted fabrics, cloth, cotton, terry cloth, foams, and polyurethane.

According to the teachings of the present invention, the chamber is configured to hold water (including aqueous solutions). In embodiments, the chamber is provided with a fluid inlet configured to couple with a fluid source. In embodiments, the fluid inlet comprises a quick-release fitting configured to couple with the fluid source, and the fluid inlet comprises a self-sealing valve configured to prevent fluid flow from the inlet if uncoupled from the fluid source. In embodiments, the one-way valve of the fluid inlet comprises a self-sealing valve.

In embodiments of the present invention, the chamber is provided with a fluid outlet configured to couple with a fluid drain. In embodiments, the fluid outlet comprises a quick-release fitting configured to couple with the fluid drain and a self-sealing valve configured to prevent fluid flow from the outlet if uncoupled from the fluid drain.

According to teachings of the present invention, the plastically deformable element comprises a metal. Suitable metals include, but are not limited to aluminum, silver, gold, copper and alloys thereof.

In embodiments of the present invention, the heat transfer component is flexible. In embodiments of the present invention, the heat transfer component is elastic. Suitable materials from which a heat transfer component is fashioned include but are not limited to elastomer, rubber, latex, neoprene, neoprene rubber, silicon and silicon rubber. In embodiments, the heat transfer component comprises latex.

According to teachings of the present invention, there is provided a device, further including a spacer component for maintaining opposing walls of the chamber in a substantially defined spaced-apart relation relative to each other. In embodiments, there is provided a device, wherein the spacer component comprises a plurality of substantially rigid elements, each of the substantially rigid elements being retained within a portion of the plastically deformable element. In embodiments, the spacer components are fastened to the opposing walls.

According to teachings of the present invention, there is provided a device, wherein each of the walls is no greater than 1 mm thick, no greater than 0.8 mm thick, or no greater than 0.5 mm thick.

According to teachings of the present invention, the heat transfer component comprises at least one heating element, the at least one heating element having a shape corresponding substantially to that of the plastically deformable element.

According to teachings of the present invention, the at least one heating element is configured to generate heat. In embodiments, the at least one heating element is intimately associated with the plastically deformable element. In embodiments, the at least one heating element is integrally formed with the plastically deformable element. In embodiments, the at least one the heating element is an electrical heating element or a chemical heating element.

According to teachings of the present invention, the heat transfer component comprises: a fluid chamber, the fluid chamber functionally associated with the deformable sheet element such that the deformable sheet element substantially defines the shape of the fluid chamber; and at least one heating element, the at least one heating element having a shape corresponding substantially to that of the plastically deformable element.

According to teachings of the present invention, the plastically deformable element is substantially a sheet having a shape, the shape selected from the group consisting of square, circular, oval, rectangular, comb-shaped, star-shaped and triangular. In embodiments, the plastically deformable element is configured to substantially wrap about a portion of a body.

According to teachings of the present invention, there is provided a mechanism for securing the device about a portion of a body. In embodiments, the device is substantially of planar topology with first and second edges, the securing mechanism including a first component proximal to the first edge, whereby, when the device is positioned about a portion of a body, the first component of the securing mechanism is brought into registration with the second edge portion, and the first securing component is actuated so as to maintain the device in position about the portion of the body. In embodiments, there is provided a securing mechanism including a second component located proximal to the second edge whereby, when the device is positioned about a portion of a body and the first component of the securing mechanism is brought into registration with the second component, the securing mechanism is actuated so as to maintain the device in position relative to the portion of the body.

According to teachings of the present invention, the device is enclosed within a sealed container. In embodiments, the device is sterilized prior to being enclosed in the sealed container. In embodiments, the container is sterilizable.

According to teachings of the present invention, the device is substantially transparent to at least one imaging modality selected from the group consisting of X-ray imaging, radiation-emission imaging and magnetic resonance imaging.

In embodiments of the present invention, there is provided a method for heating a vessel of injectable fluid (e.g., an infusion bag such as of saline or blood), the method comprising wrapping a vessel of injectable fluid with a device of the present invention and activating the heating element so as to heat the injectable fluid.

Additionally according to teachings of the present invention, there is provided a kit for the controlled heating or cooling of a portion of a body, comprising: at least two devices for the controlled heating or cooling of a portion of the body, each the device including: a contact surface; a plastically-deformable, substantially sheet element configured to define the shape of the contact surface; and a heat transfer component, associated with the plastically deformable element, for adjusting the temperature of the contact surface.

In embodiments of the present invention, the kit further comprises a component for exchanging energy with the heat transfer components of the devices. In embodiments, the component for exchanging energy comprises: at least one inlet conduit associated with each of the devices, for providing a flow of fluid into the devices; at least one outlet conduit associated with each of the devices, for accepting a flow of fluid out of the devices; and a control unit for controlling the temperature of the heat transfer components.

In embodiments, the inlet conduits connect the heat transfer components to a fluid source and the plurality of outlet conduits connect the heat transfer components to a fluid disposal point, and the control unit controls the temperature of fluid flowing via the plurality of inlet conduits into the heat transfer components, thereby controlling the temperature of the heat transfer components.

In embodiments, there is provided a kit, the heat transfer component of each the device comprising at least one electrical heating element further comprising: a controllable electrical power source for providing electricity to the electrical heating elements; and a control unit for controlling the amount of power provided by the power source to the electrical heating elements, thereby controlling the temperature of the heat transfer components.

In embodiments, a shape of an electrical heating element substantially corresponds to that of a respective the plastically deformable element. In embodiments, an electrical heating element is integrally formed with the plastically deformable element of a respective device. In embodiments, the heat transfer component comprises at least one chemical heating element, wherein the at least one chemical heating element includes a controllable chemical source for initiating a chemical reaction to change the temperature of a respective heat transfer component.

According to teachings of the present invention, there is provided a self-sealing valve fabricated of a flexible material, the valve defined by a cylindrical portion of the flexible material, the valve having an inner surface and an outer surface, the inner surface defining a passageway through the valve, the passageway having an inlet end and an outlet end, the valve characterized in that, fluid flowing through a tubular element inserted into the passageway at the inlet end travels from the inlet end towards the outlet end and when no fluid flows through the tubular element towards the inlet, a pressure differential between a fluid on the outer surface at the outlet end and a fluid inside the passageway forces the passageway to be closed, thereby preventing a flow of fluid from the outlet end towards the inlet end. In embodiments of the present invention, the flexible material is rubber. In embodiments, the valve is formed substantially entirely of latex or similar material.

According to the teachings of the present invention, there is also provided a valve substantially of two strips of flexible material conjoined at two sides so as to substantially define a flattened tube. As a self-sealing intake valve, a fluid source is placed between the two strips at the upstream end of the flattened tube where the downstream end of the valve protrudes into the bulk of a fluid. Fluid flows from the fluid source into the flattened tube, pushing apart the two strips and flowing out the downstream end of the flattened tube. Due to the pressure applied by the bulk of the fluid onto the downstream end of the flattened tube (and preferably also a self-affinity of the material), fluid cannot back-flow through the downstream end of the tube. Further, if the downstream end of the flattened tube is immersed in a fluid, the fluid pressure keeps the two strips pressed tightly together ensuring a seal. As a self-sealing outlet valve, a fluid drain is placed between the two strips from the downstream end of the flattened tube to proximity with the upstream end of the flattened tube so that the fluid drain maintains the two strips at the upstream end separated and non-sealed where the upstream end of the valve protrudes into the bulk of a fluid. In such a position, fluid can freely drain from the upstream end of the flattened tube through the fluid drain out through the downstream end of the flattened tube as long as the fluid drain is in position. When the fluid drain is withdrawn whether by accident or by design, out through the downstream end to the extent that the fluid drain does not maintain the two strips at the upstream end separated, then the pressure applied by the bulk of the fluid onto the upstream end of the flattened tube (and preferably also a self-affinity of the material), closes off the tube preventing further fluid flow through the tube.

According to teachings of the present invention, there is also provided a method for heating or cooling at least a portion of the body of a person, comprising the steps of: (a) providing a device having: a contact surface; a plastically deformable, substantially sheet element configured to define the shape of the contact surface; and a heat transfer component, associated with the plastically deformable element, for adjusting the temperature of the contact surface; (b) positioning the device relative to a portion of a body of a person and manipulating the plastically deformable element such that the contact surface of the device is substantially in contact with the portion of the body; and (c) exchanging energy with the heat transfer component of the device so as to change the temperature of the heat transfer component, thereby heating or cooling at least a portion of the body.

In embodiments of the present invention, the exchanging energy includes providing electrical power to an electrical heating element functionally associated with the heat transfer component.

In embodiments, the device further comprises a chamber having walls of a flexible material including a fluid, and the electrical heating element heats the fluid. In embodiments, the device further comprises a chamber having walls of a flexible material, a fluid inlet and a fluid outlet; and wherein the exchanging energy includes providing a flow of fluid through the chamber in the inlet and out the outlet. In embodiments, the fluid is at a temperature higher than a portion of the body so as to affect heating of the portion. In embodiments, the fluid is at a temperature lower than a respective portion of the body so as to effect cooling of that portion.

In embodiments of the present invention, the exchanging energy includes initiating a chemical reaction that changes the temperature of the heat transfer component.

In embodiments, the chemical reaction is exothermic. In embodiments, the chemical reaction is endothermic.

In embodiments of the present invention, the device is substantially of planar topology with a first and second edge portion, the step (b) including plastically deforming the device around the body portion until the edge portions are proximally disposed. In embodiments, between the first and second edge portions there is a gap allowing access to a part of a surface of the body portion. In embodiments, the first and second edge portions substantially abut so as to substantially wrap the body portion with the device. In embodiments, the first and second edge portions substantially overlap as to wrap the body portion with the device. In embodiments, the overlapping is of no more than about 180 degrees, of no more than about 90 degrees, of no more than about 45 degrees, and of no more than about 20 degrees.

In embodiments of the present invention, there is provided a method including the additional step (for example in between steps (b) and (c)) of providing a mechanism for securing the device to the portion of the body.

In embodiments, the device further includes a mechanism for securing the device about a portion of a body. In embodiments, the securing mechanism includes a first component proximal to the first edge, whereby, when the device is positioned about a portion of a body, the first component of the securing mechanism is brought into registration with the second edge portion, the first securing component is actuated so as to maintain the device in position about the portion of the body. In embodiments, the securing mechanism includes a second component located proximal to the second edge whereby, when the device is positioned about a portion of a body and the first component of the securing mechanism is brought into registration with the second component, the securing mechanism is actuated so as to maintain the device in position relative to the portion of the body.

In embodiments of the present invention, the portion of the body is selected from the group consisting of hand, arm, forearm, upper arm, foot, leg, lower leg, thigh, abdomen, chest, neck and head.

In embodiments, there is provided a method further comprising step (d) while the device is positioned relative to the portion of the body, imaging the portion using a medical imaging modality selected from the group consisting of X-ray imaging, radiation emission imaging and magnetic resonance imaging.

According to teachings of the present invention, there is also provided a method for manufacturing a device for the controlled heating or cooling of a portion of a body, the method comprising the steps of: (a) providing a plastically deformable, substantially sheet element including a plurality of apertures; (b) dipping the plastically deformable element in a bath of uncured polymer (such as rubber, preferably latex rubber) to coat the plastically deformable element with a polymer film; and (c) curing the uncured polymer so as to form a flexible, walled chamber in which the plastically deformable element is contained. In the case of rubber, curing comprises vulcanization.

In embodiments, the method further comprises, in between steps (a) and (b): (d) dipping the plastically deformable element in at least one additional bath of uncured polymer to coat the plastically deformable element with at least one additional polymer film. In embodiments, the at least one additional bath of uncured polymer in step (d) and the bath of polymer rubber in step (b) contain different compositions so that each polymer film results in a cured product having somewhat different properties.

In embodiments of the present invention, the method further comprises, in between steps (a) and (b): (d) blunting the outer edges of the plastically deformable element. In embodiments, the blunting of edges comprises folding over the edges of the plastically deformable element.

In embodiments, the method further comprises, in between steps (a) and (b): (d) blunting the edges of the apertures. In embodiments, the blunting of aperture edges comprises placing a grommet in an aperture.

In embodiments of the present invention, the plastically deformable element includes a first tab and wherein, during the dipping, the plastically deformable element is held at the first tab by a holder. In embodiments, the holder comprises a fluid inlet and the method further comprises: (e) subsequent to (c), forcing fluid through the fluid inlet into the chamber thereby determining the integrity of walls of the chamber.

In embodiments of the present invention, the plastically deformable element comprises an electrical heating element. In embodiments, the plastically deformable element includes a first tab, wherein during the dipping, the plastically deformable element is held at the first tab by a holder, the method further comprising: (f) detaching the first tab from the holder so as to define an opening into the chamber; and (g) providing an electrical connector through the opening to allow provision of the electrical heating element with electrical power. In embodiments, the method further comprises: (f) detaching the first tab from the holder so as to define an opening into the chamber.

According to embodiments of the present invention, the plastically deformable element includes a second tab, the method further comprising: (h) subsequent to (c), cutting of a portion of cured polymer from the second tab so as to define an opening into the chamber. In embodiments, the method further comprises placing connectors in the openings so as to define a fluid inlet into and a fluid outlet out of the chamber. In embodiments, the connectors comprise unidirectional valves or self-sealing valves.

According to embodiments of the present invention, the method further comprises associating with at least a portion of the outside of the chamber an outer thermoinsulating layer. In embodiments, the method further comprises associating with at least a portion of the outside of the chamber an outer hypoallergenic insulating layer. In embodiments, the method further comprises associating with at least a portion of the outside of the chamber a securing component.

In embodiments of the present invention, the method further comprises (i) subsequent to (c), sterilizing the chamber. In embodiments, the sterilizing comprises exposing the chamber to gamma radiation. In embodiments, the sterilizing comprises exposing the chamber to ethylene oxide. In embodiments, the method comprises, subsequent to (c), placing the chamber in a sterilizable packaging.

In embodiments, a device of the present invention manufactured in accordance with the above comprises a plastically deformable sheet element encased in a flexible material. In such cases, the sheet element is generally provided with a tab that is held or grasped to allow dipping of the sheet element into the polymer bath. Such a tab generally remains at least partially uncoated, providing an opening allowing attachment of connectors, wires and the like necessary for operation of a heating element, for example an electrical heating element. Generally, subsequent to attachment such an opening is closed (for example with a clamp or tie) or sealed (for example by welding or adhesive).

In embodiments, a device of the present invention manufactured in accordance with the above comprises a plastically deformable sheet element encased in a flexible material constituting a chamber in which a chemical compound (for heating or cooling) or a fluid is held. In such cases, the sheet element is generally provided with a tab that is held or grasped to allow dipping of the sheet element into the polymer bath. Such a tab generally remains at least partially uncoated, providing an opening allowing attachment of connectors, wires and the like necessary for operation of a heating element, for example an electrical heating element, for filling the chamber with a fluid such as water (or aqueous solution, or for filling the chamber with chemical compounds used for heating or cooling. Generally, subsequent to attachment and filling such an opening is closed (for example with a clamp or tie) or sealed (for example by welding or adhesive).

In embodiments, a device of the present invention manufactured in accordance with the above comprises a plastically deformable sheet element encased in a flexible material constituting a chamber through which a fluid is allowed to flow, necessitating at least one inlet and at least one outlet (preferably no more than one inlet and one outlet). In such cases, the sheet element is generally provided with a tab that is held or grasped to allow dipping of the sheet element into the polymer bath. Such a tab generally remains at least partially uncoated, providing an opening that, upon appropriate finishing such as addition of a valve, constitutes a first inlet or an outlet. A second opening is then produced by cutting the cured polymer. Preferably, the plastically deformable sheet element is provided with a second tab that generally is coated with polymer subsequent to dipping and polymer is cut from about the tab to provide an opening that that, upon appropriate finishing such as addition of a valve, constitutes a second inlet or an outlet.

According to teachings of the present invention, there is also provided a method of producing a self-sealing valve, the method comprising the steps of: (a) providing a substantially planar elongated former; (b) while holding the former by a proximal end, dipping the former in a bath of uncured polymer (such as rubber, preferably latex rubber) to coat the former with an uncured polymer film; (c) curing the uncured polymer so as to form a flattened, flexible-walled cylinder of cured polymer; (d) removing the cylinder from the former; and (e) cutting an aperture in a portion of cured polymer film; thereby forming a flexible walled flattened tube that constitutes a self-sealing valve.

According to embodiments, there is provided a method, wherein step (d) is performed prior to performing step (e). In embodiments, step (e) is performed subsequent to performing step (d).

Generally, a self-sealing valve must have at least two apertures. In embodiments, one aperture is formed by cutting of the flattened tube while another aperture is formed by the border of where the former was dipped in the polymer bath. In embodiments, two or more apertures are cut in the flattened tube, for example when the former is entirely coated by polymer. Apertures can be, for example, slits, holes, perforations or fenestrations anywhere on the flattened tube including on the top, bottom, sides or ends of the flattened tube.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural aspects of the invention in more detail than is necessary for a fundamental understanding of the invention, the description taken with the drawings making apparent to those skilled in the art how several forms of the invention may be embodied in practice.

In the drawings:

FIG. 1 is a pictorial illustration of a plastically deformable, substantially sheet element used for the production of a device for the controlled heating or cooling of a portion of the body in accordance with an embodiment of the present invention;

FIG. 2 is a cross-sectional illustration of the plastically deformable element of FIG. 1, taken in the direction of line A-A therein;

FIG. 3 is a pictorial illustration of the device for the controlled heating or cooling of a portion of the body, wherein the unidirectional flow of fluid through the device is shown;

FIG. 4 is an enlarged view of a portion of a device shown in FIG. 3, taken in the direction of line A-A therein;

FIG. 5 is a pictorial illustration of the device of FIG. 3, wherein the device has been deformed so as to conform to the shape of a portion of the body;

FIG. 6 is a pictorial illustration of the device of FIG. 5, wherein securing means is shown;

FIGS. 7-10 are pictorial illustrations of a portion of the device of FIG. 5 employing a self-sealing valve according to an embodiment of the present invention;

FIGS. 11A-F is a pictorial illustration of a device for the controlled heating or cooling of a portion of the body in accordance with an embodiment of the present invention, wherein the device is configured so as to correspond to a particular portion of the body;

FIGS. 12-14 is a pictorial illustration showing a patient for whom at least two adjacent devices are employed, in accordance with an embodiment of the present invention;

FIG. 15 is a pictorial illustration of a kit for the controlled heating or cooling of a portion of the body in accordance with an embodiment of the present invention;

FIG. 16 is a pictorial illustration of a kit for the controlled heating or cooling of a bed;

FIGS. 17-20 are pictorial illustrations of devices for the controlled heating or cooling of portions of the body in accordance with further embodiments of the present invention;

FIG. 21 is a pictorial illustration of a device for the controlled heating of a portion of the body in accordance with a further embodiment of the present invention;

FIG. 22 is an enlarged cross-sectional illustration of the device of FIG. 21, taken in the direction of lines A-A therein; and

FIGS. 23A-D are pictorial illustrations of a self-sealing valve according to an embodiment of the present invention.

### DESCRIPTION OF EMBODIMENTS OF THE INVENTION

An aspect of the present invention is a device for the controlled heating or cooling of a portion of the body comprising: a contact surface; a plastically deformable, substantially sheet element configured to define the shape of the contact surface; and a heat transfer component, associated with the plastically deformable element for adjusting the temperature of the contact surface. According to an embodiment of the present invention there is provided a kit for the controlled heating or cooling of a portion of the body, the kit comprising: at least two devices for the controlled heating or cooling of a portion of the body, substantially as described above.

An aspect of the present invention is a self-sealing valve substantially of two strips of flexible material conjoined at two sides so as to substantially define a flattened tube.

An aspect of the present invention is of a method for heating or cooling at least a portion of the body of a person, the method comprising the steps of: (a) providing a device having: a contact surface; a plastically deformable, substantially sheet element configured to define the shape of the contact surface; and a heat transfer component, associated with the plastically deformable element, for adjusting the temperature of the contact surface; (b) positioning the device about a corresponding portion of the body of the person and manipulating the plastically deformable element such that the contact surface of the device is substantially in contact with the portion of the body; (c) exchanging energy with the heat transfer component of the device so as to change the temperature of the heat transfer component thereby heating or cooling the portion of the body.

An aspect of the present invention is a method for manufacturing a device for the controlled heating or cooling of a portion of the body, the method comprising the steps of: (a) providing a plastically deformable, substantially sheet element; (b) dipping the plastically deformable element in a bath of uncured polymer to coat the plastically deformable element with a polymer film; and (c) curing the uncured polymer so as to form a flexible walled chamber in which the plastically deformable element is contained.

An aspect of the present invention is a method of producing a self-sealing valve, the method comprising the steps of: (a) providing a substantially planar former; (b) dipping the former in a bath of uncured polymer to coat the former with an uncured polymer film; (c) curing the polymer coating the former; and (d) cutting a portion of the cured polymer material coating the former, at least one location to provide an inlet and an outlet.

The principles and uses of the teachings of the present invention may be better understood with reference to the accompanying description, figures and example. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of chemistry, engineering, material sciences and physics. Such techniques are thoroughly explained in the literature.

Unless otherwise defined, all technical and scientific terms used herein have the same meaning as commonly understood by one of ordinary skill in the art to which the invention belongs. In addition, the descriptions, materials, methods, and examples are illustrative only and not intended to be limiting. Methods and materials similar or equivalent to those described herein can be used in the practice or testing of the present invention.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Implementation of the methods of the present invention involves performing or completing selected tasks or steps manually, automatically, or a combination thereof.

The term “plastic” and grammatical variants thereof is to be understood as being deformable without rupture by the application of force, preferably reversibly deformable, into a substantially desired shape and substantially retaining the desired shape when the force is released.

Aspects of the present invention provide a device for and method of controlled heating or cooling of at least a portion of the body using a device which, in embodiments, is sterilizable, disposable, inexpensive to produce, and simple to operate. Generally, embodiments of the present invention are based on the use of a temperature regulating device having a contact surface; a plastically deformable, substantially sheet element; and a heat transfer component, associated with the plastically deformable element, for adjusting the temperature of the contact surface. When a device is used, the plastically deformable element conforms to a portion of the body and the heat transfer component adjusts the temperature of the contact surface of the device, as desired, thus enabling the controlled heating or cooling of the portion of the body.

In embodiments of the present invention the heat transfer component of the device comprises a waterproof chamber through which fluid such as water or water solution at a desired temperature flows. A control unit may be attached to the device, by means of a system of inlet and outlet conduits, whereby the fluid at the desired temperature flows through the device. The device is preferably constructed so as to allow the quick and simple attachment of conduits into the device. In embodiments, self-sealing valves are included so as to provide the device with leak-proof seals at the inlet and outlet of the device.

In embodiments of the present invention, the heat transfer component comprises an electrical heating element provided with a control unit for adjusting the temperature of the contact surface.

In embodiments of the present invention, the device may be configured so as to conform to a specific portion of the body and may be secured thereto by a securing mechanism provided. If desired, at least two devices corresponding to different portions of the body may be secured together to be utilized simultaneously as a kit or as a complete suit.

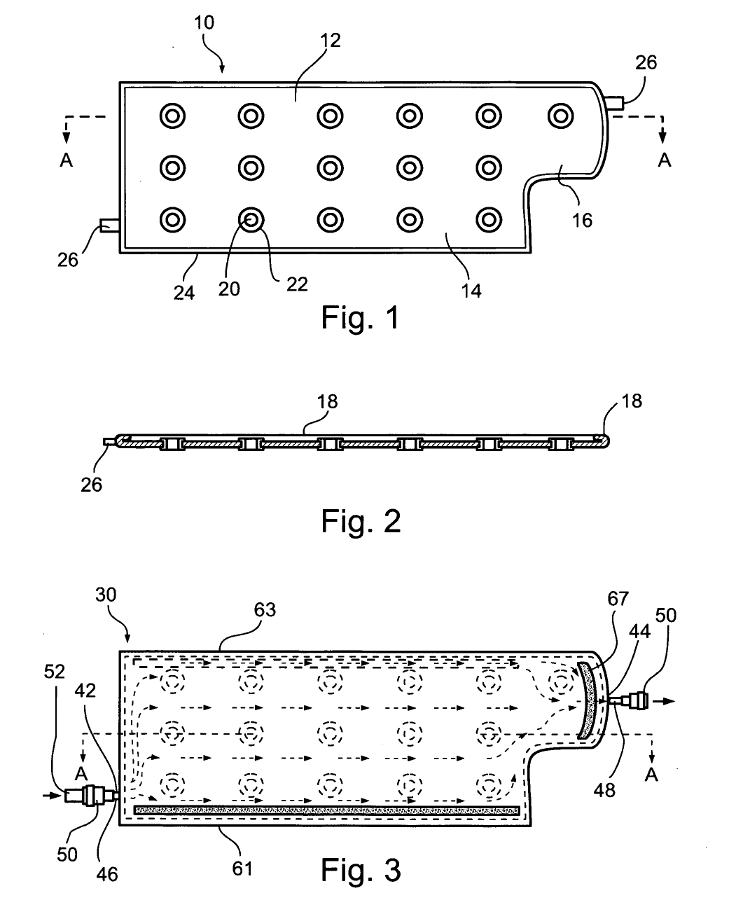

With reference to the drawings, in FIGS. 1 and 2 there is shown a plastically deformable, substantially sheet element 10 included in a device for the controlled heating or cooling of a portion of the body in accordance with an embodiment of the teachings of the present invention. In the embodiment shown, element 10 is defined by a substantially planar lamina 12 configured to conform to a portion of the body. In embodiments of the present invention the plastically deformable element 10 comprises, substantially comprises, essentially consists or even consists of a metal, e.g., aluminum, silver, gold, copper and alloys thereof. Lamina 12 defines a rectangular area 14 with a portion 16 extending out of rectangular area 14. While, in the embodiment described element 10 is formed of aluminum, it will be appreciated by persons skilled in the art that, if desired, any other suitable plastically deformable material may be employed such as discussed above.

The outer edges 18 of element 10 have been folded under lamina 12, such that there are no sharp edges along the periphery 24 of lamina 12. Element 10 is provided with a plurality of apertures 20 through each of which there is disposed a spacer component defined by a grommet 22. Element 10 is also provided with a pair of aluminum tabs 26 which extend from the periphery 24 of lamina 12. The functions of grommets 22 and tabs 26 will be discussed in detail below.

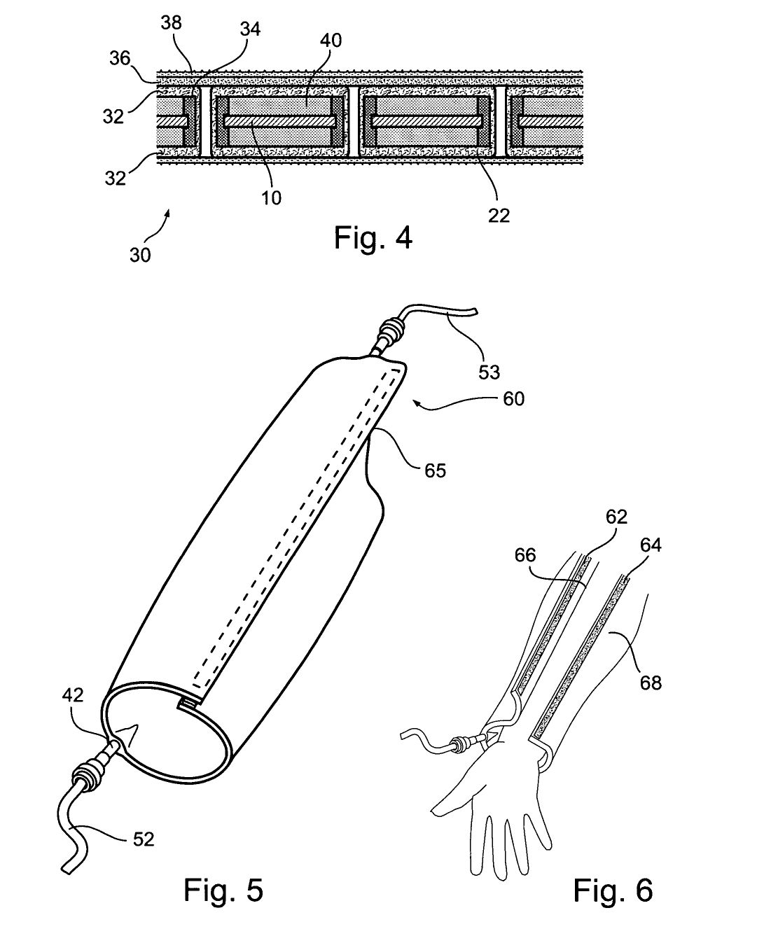

FIG. 3 shows a device 30 for the controlled heating or cooling of a portion of the body, in accordance with embodiments of the present invention. As more clearly detailed in FIG. 4, device 30 is formed of element 10 (FIG. 1) having a layer 32 of latex material disposed on either side of lamina 12. Latex layers 32 extend around the periphery 24 of lamina 12, through grommets 22, and about tabs 26 such that layers 32 form a seamless, waterproof chamber 40 about plastically deformable element 10. The waterproof chamber 40 defines a heat transfer component of device 30, which will be discussed in detail below. A surface 34 of latex layer 32 is preferably provided with a layer 36 of polyurethane elastomer sponge and an adjacent layer 38 of terry cloth. If desired, a second layer 38 of terry cloth may be provided on the outer surface of the latex layer 32 distal to the layer 36.

As shown in FIG. 4, device 30 defines a layered structure having an upper layer 38 of terry cloth, a layer 36 of polyurethane elastomer sponge, an upper latex layer 32, waterproof chamber 40 in the middle of which is disposed an inner layer 10 of aluminum, a lower latex layer 32, and an optional outer layer 38 terry cloth.

In embodiments of the present invention the heat transfer component comprises, substantially comprises, essentially consists or even consists of a material selected from the group consisting of elastomer, rubber, latex, silicon, neoprene, neoprene rubber or silicon rubber. While the device 30 is described as having layers 32 formed of latex material, it will be appreciated by persons skilled in the art that, if desired, any suitable flexible material especially a polymeric material, such as discussed above, may be used to form chamber 40. Desired properties of such a flexible material include elasticity, adhesiveness, sterlizability, and puncture resistance. Advantages of latex include low cost, ease of manufacture and widely accepted use in the field of medicine.

It will be appreciated by persons skilled in the art that, due to the particular constructions of the embodiment of the present invention as depicted in FIGS. 3 and 4, whereby the device is substantially made of latex and aluminum, the device is substantially transparent to various medical imaging modalities such as X-ray imaging, radiation-emission imaging and magnetic resonance imaging.

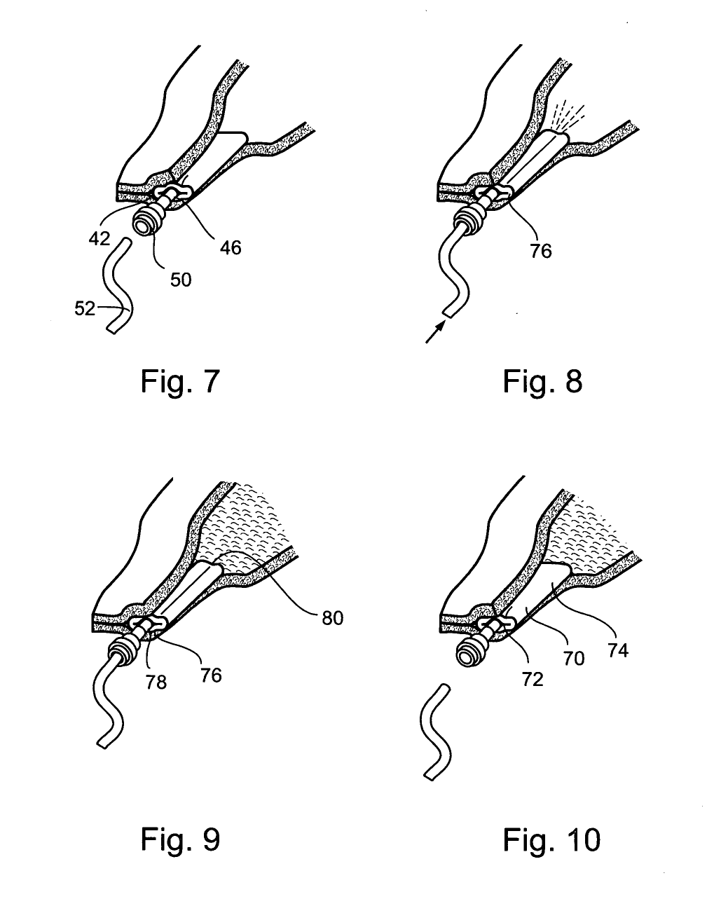

The portions of latex layers 32 that are disposed about tabs 26 have been removed, thus providing chamber 40 of device 30 with an inlet end 42 and an outlet end 44. Inlet end 42 is provided with an inlet conduit 46 that is inserted thereinto. Similarly, outlet end 44 is provided with an outlet conduit 48 that is inserted thereinto. Inlet conduit 46 and outlet conduit 48 are attached to fittings 50 that connect, via source conduit 52 and disposal conduit 53 (FIG. 5), to a source of fluid (not shown) and to a fluid disposal point (not shown), respectively. When in use, fluid is caused to flow into chamber 40 via inlet conduit 46 and out of chamber 40 via outlet conduit 48, thus providing device 30 with a unidirectional flow of fluid, as shown in FIG. 3. In order to ensure the unidirectional flow of fluid, device 30 may be provided with a self-sealing valve such as, for example, valve 70, which will be described below with reference to FIGS. 7-10 and 23A-D.

Embodiments of the present invention provide a device for the controlled heating or cooling of a portion of the body. When in use, if it is desired to heat a portion of the body, fluid at a desired temperature higher than the body temperature, is caused to flow through the chamber of the device and the device brought into contact with the portion of the body such that heat is transferred from the device to the body portion. Alternatively, if it is desired to cool a portion of the body, fluid at a desired temperature lower than the temperature of the body portion, is caused to flow through the chamber of the device and the device is brought into contact with the portion of the body, such that heat from the body is transferred to the device.

The novel construction of the device with its fluid chamber allows safe heating or cooling a body portion of a person who may be unconscious, without the risk of burning the body portion. The heating or cooling effect of the device may be maintained for a long period of time such as, for example, on a patient during a surgical procedure, and while there is pressure and weight of the patient on the device.

While embodiments of the present invention may employ any suitable fluid within the chamber 40, it is preferable to utilize water (or aqueous solutions) that is inexpensive, readily available, nontoxic, and has a high heat capacity.

While the embodiment of the device described with reference to FIGS. 3-4 is shown as having a plastically deformable element 10 disposed within a waterproof chamber 40, it will be appreciated by persons skilled in the art upon perusal of the description herein that, if desired, the plastically deformable element may be located outside the waterproof chamber and adjacent thereto, without departing from the scope of the invention.

In use, for example, in the heating of an arm, the device 30 may first be placed under the arm such that the arm rests on layer 38, approximately in the center of device 30, and such that the arm lies approximately parallel to edges 61 and 63 of device 30. Then device 30 may be deformed by moving edges 61 and 63 around the arm until they are nearly abutting. An advantage of this use of the device is that a gap may be left in between edges 61 and 63 of device 30, thereby allowing access to the arm as may be needed, for example, in order to administer an injection. The elastic properties of the device 30 enable edges 61 and 63 to be moved closer together, if desired, until they abut or even overlap, so as to enable more complete coverage of the arm and thus providing more efficient heating to the arm. It will be appreciated by persons skilled in the art that the amount of overlap of the edges 61 and 63 of the device 30 around the arm may be up to 180° without physically moving the arm.

With additional reference to FIGS. 5 and 6, there is shown device 30 that has been deformed so as to conform to the arm of a patient, such that it surrounds the arm and contact surface 66 of latex layer 32 is substantially in contact with the skin of the patient's arm. While the aluminum construction of lamina 12 allows device 30 to remain in its deformed configuration about the arm of the patient, device 30 may be provided with additional means for maintaining its configuration if, for example, movement of the patient would otherwise allow the arm to be moved out of contact with device 30. For this purpose, device 30 has been provided with securing means 60 which may comprise, for example, opposing “hook and loop” fastener strips (e.g., Velcro®, Velcro Industries N.V.) defining first and second securing elements 62 and 64. First and second securing elements 62 and 64 are disposed on respective surfaces 66 and 68 of latex layer 32, proximal to the periphery 65 thereof. Thus, when the arm of a patient is placed on device 30 and device 30 has been deformed so as to conform to the arm, first securing element 62 may be brought into registration with second securing element 64 so as to maintain device 30 in position about the arm of the patient.

Alternatively, either or both of surfaces 66 and 68 may be provided with an adhesive strip whereby when surfaces 66 and 68 are brought into registration, the adhesive strip will maintain contact between the surfaces, thus maintaining device 30 in position about the arm of the patient.

Embodiments of the present invention provide a device that may be quickly and easily fitted onto a portion of the body with complete contact between the device and the body portion for effective heating or cooling of the body portion.

When it is desired to heat or cool a portion of the body, a device having a configuration corresponding to the specific body portion is brought into contact with that portion of the body and may be secured thereto by any suitable mechanism, such as discussed above. Water at the desired temperature is caused to flow into the waterproof chamber 40 via inlet conduit 46 and out of chamber 40 via outlet conduit 48, thus providing device 30 with a unidirectional flow of water, as shown in FIG. 3. Depending on the water temperature chosen, the flow of water through the device allows the portion of the body in contact with the device to be heated or cooled by being in contact with chamber 40, which defines the heat transfer component of the device.

As shown in FIG. 3, device 30 may be also provided with an additional securing element 67 which may be, for example, an adhesive strip, such that device 30 may be secured to another device (not shown) for heating or cooling of another body portion, adjacent to the arm around which device 30 is secured. The simultaneous use of at least two separate devices for the controlled heating or cooling of portions of the body is discussed further below, with reference to FIGS. 11A-15.

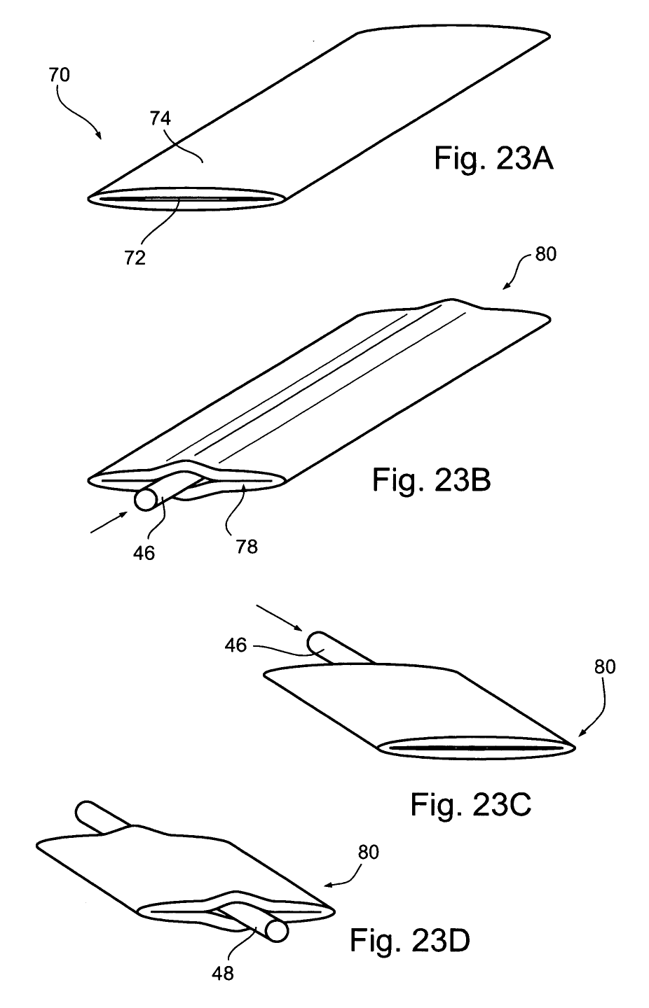

Referring now to FIGS. 7-10, there is shown a portion of device 30 for the controlled heating or cooling of a portion of the body, in accordance with an embodiment of the present invention, wherein inlet end 42 of device 30 has been provided with a self-sealing valve 70. Valve 70 is constructed of a single piece of a elastic material such as latex, as will be discussed below with reference to FIGS. 23A-D, and has a generally cylindrical configuration. Valve 70 includes an inner surface 72 and an outer surface 74, the inner surface 72 defining a passageway 76 through valve 70, the passageway 76 having an inlet end 78 and an outlet end 80. Left undisturbed, valve 70 will remain in a closed configuration, as shown in FIG. 23A. While valve 70 may be employed as either an inlet valve or an outlet valve for a device in accordance with an embodiment of the present invention, the performance of valve 70 will depend on its function as either an inlet valve or an outlet valve, as will be discussed below.

When employed at inlet end 42 of a device in accordance with an embodiment of the present invention, valve 70 may be opened by the insertion of an inlet conduit 46 into passageway 76, as shown in FIG. 23B, whereat inlet conduit 46 is retained inside passageway 76 by the self-affinity properties of elastic material of valve 70. A particular characteristic of valve 70 is that, due to its construction, the material of the valve 70 will form a waterproof seal around inlet conduit 46. Valve 70 at inlet end 42 will remain open as long as fluid flows through inlet conduit 46.

While valve 70 has been described as being employed at an inlet end 42 of device 30, it will be appreciated by persons skilled in the art upon perusal of the description herein that valve 70 may be similarly employed at an outlet end of a device in accordance with an embodiment of the present invention, so as to provide a self-sealing valve thereat.

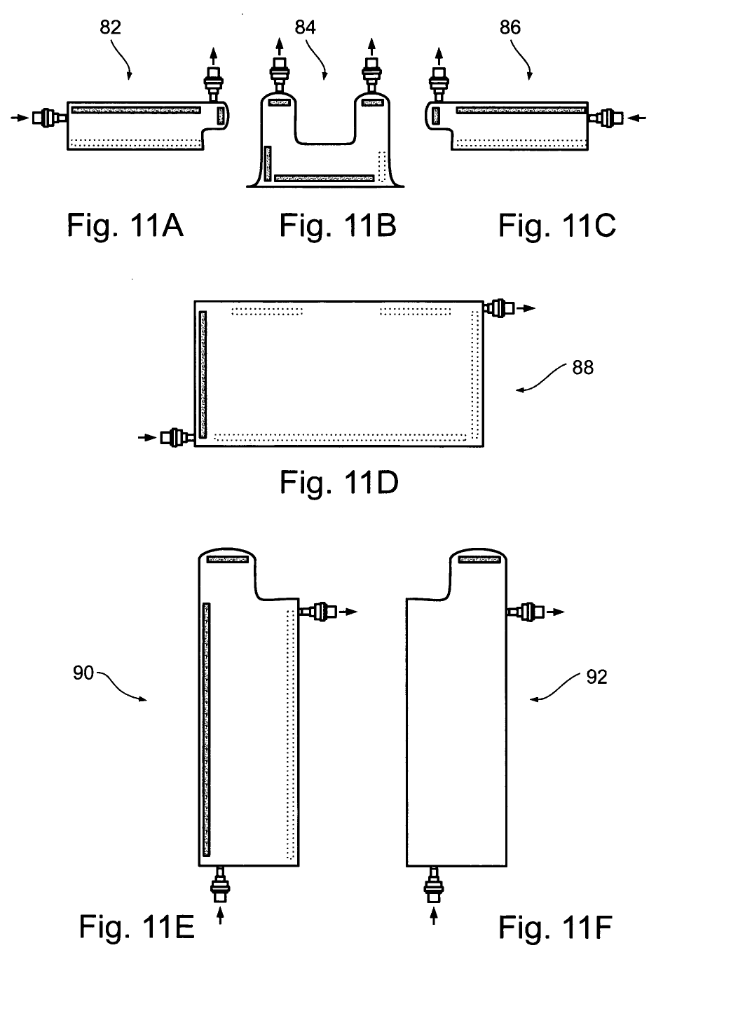

Device 30 may be provided with a quick-release fitting 50 (FIG. 7), as known in the art and available commercially, such that conduit 52 may be inserted thereinto in order to channel the flow of water from a source (not shown), via passageway 76, and into chamber 40 (FIGS. 8-9). As chamber 40 becomes filled, if the pressure of the water flow is reduced, self-sealing valve 70 will close, as shown in FIG. 23C, thereby preventing water from flowing back therethrough. Additionally, in the event that conduit 52 becomes detached from fitting 50 (FIG. 10), self-sealing valve 70 will close, and will thus prevent any water from flowing out of chamber 40.

When employed at the outlet end 80 of a device in accordance with an embodiment of the present invention, valve 70 may be opened by the insertion of an outlet conduit 48, as shown in FIG. 23D, whereat outlet conduit 48 is retained inside passageway 76 by the adhesive properties of elastic material of valve 70. A particular characteristic of valve 70 is that, due to its construction, the material of the valve 70 will form a waterproof seal around outlet conduit 48.

It should be noted that, with regard to inlet end 78 of valve 70, inlet conduit 46 needs to be only partially inserted into the inlet end 78 of valve 70, as the pressure of the fluid flowing through inlet conduit 46 will keep valve 70 open at the inlet end 78 thereof. In contrast, in order to keep valve 70 open at the outlet end thereof, outlet conduit 48 must be inserted completely through valve 70 (FIG. 23D) in order to prevent valve 70 from closing.

Device 30 thus has a simple means for quickly and efficiently connecting to a fluid source and to a fluid disposal point which does not require complex or expensive connections between the device and the fluid source or between the device and the fluid disposal point.

Valve 70 may be manufactured by any method known in the art, for example, by coating a generally planar, former, such as made of aluminum foil or plate, with an uncured polymer such as rubber such as latex rubber. Once the polymer is cured, the ends of the cured polymer may be cut off and the former removed, thus producing a self-sealing valve 70.

Each of FIGS. 11A-F illustrates a device for the controlled heating or cooling of a portion of the body in accordance with an embodiment of the present invention, wherein the device is configured so as to correspond to a particular portion of the body. Each of the devices shown in FIGS. 11A-F is constructed, as discussed above, so as to include all of the elements of device 30 (FIG. 3), and is operable so as to facilitate the controlled heating or cooling of a portion of the body. The devices 82, 84, 86, 88, 90, and 92 shown are specifically configured such that they are suitable for the heating or cooling of a right arm, the shoulders and upper back portion, the left arm, the torso, the right leg, and the left leg, respectively.

With regard to the embodiments shown in FIGS. 11A-F, it may be noted that the inlet and outlet of each device is disposed at opposite ends thereof. It should be understood that, in accordance with an embodiment of the present invention, it is desirable to provide each device with an evenly heated or cooled contact surface, thus providing more efficient heating or cooling of the portion of the body which is to be heated or cooled by the device. Therefore, depending on the particular shape of the device, it may be preferable to position the inlet and outlet of that device at alternate locations along the periphery of the device, so as to provide the waterproof chamber with a more efficient flow of water therethrough.

Additionally, it may be noted that the locations of the inlets and the outlets provided in each of the devices shown in FIGS. 11A-F are chosen such that, when at least two of the devices are utilized simultaneously on the body of a patient, the waterproof chambers of the devices in use may be connected by means of connecting conduits, as will be discussed below, for the controlled heating or cooling of at least two portions of the body, without conduits or fittings interfering with each other.

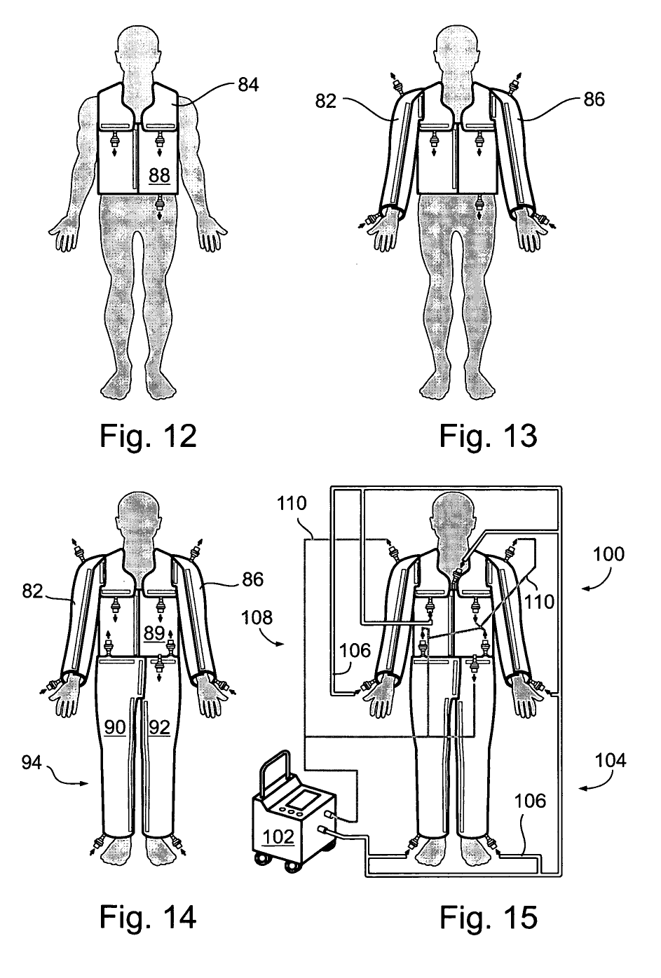

Each of FIGS. 12-14 shows a patient for whom a kit, comprising at least two devices, is employed, in accordance with an embodiment of the present invention. In FIG. 12, there are employed device 88 for the torso and device 84 for the shoulders, upper chest, and upper back portion of the body. In FIG. 13, there are employed devices 88 and 84 and the additional devices 82 and 86 for the right and left arms, respectively. FIG. 14 shows a patient on whom kit 94 is positioned, such that substantially all of the patient's torso, arms and legs may be simultaneously heated or cooled in accordance with an embodiment of the present invention. Kit 94 includes a suit comprising devices 82, 84, 86, 88, 90, and 92.

It may be noted that, while each device is constructed so as to provide heating or cooling for a particular portion of the body, in order to provide adjacent body portions with more efficient heating or cooling, it is preferable to avoid exposing portions of the body in between adjacent devices. Thus, device 84 has been configured so as to overlap device 88, so as to avoid gaps therebetween, and there has been provided suitable securing means such as, for example, “hook and loop” fastener strips or adhesive strips, discussed above with reference to FIGS. 5-6. Similarly, arm devices 82 and 86 have been configured so as to overlap device 84, and leg devices 90 and 92 have been configured so as to overlap torso device 88.

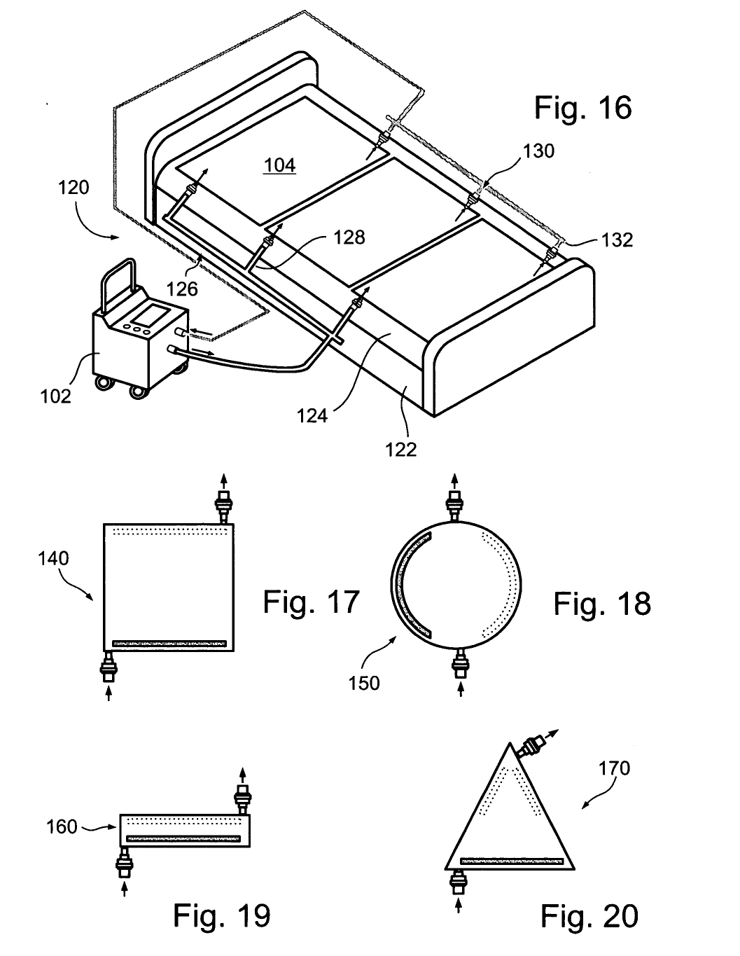

The provision of a plurality of devices, each configured to correspond to a specific portion of the body, as discussed above with reference to FIGS. 5-6 and 12-14, facilitates the controlled heating or cooling of any portion or portions of the body while providing complete coverage of the specific body portion or portions. In contrast to prior art devices, embodiments of the present invention generally provides a better fitting device having better body coverage, while requiring only a limited number of sizes of devices of varying shapes (leg, arm, torso, upper back/shoulders/upper chest). Furthermore, due to the fact that embodiments of the device of the present invention generally may have any configuration desired, such as, for example, any one of those described with reference to FIGS. 17-20, the present invention generally provides a means for facilitating the controlled heating or cooling of virtually any portion of the body or of virtually any object (FIG. 16).

While various devices corresponding to portions of an adult body have been discussed above with reference to the drawings, it should be understood that the present invention may include any other configuration of the device. For example, a device may be constructed, in accordance with the teachings of the present invention, to be employed for the heating or cooling of a portion, e.g., half of an arm or leg of an adult. This same device may be employed for the heating or cooling of the arm or leg of a small child.

Due to the substantially flat structure of embodiments of the device of the present invention and due to the modular configuration of embodiments of the present invention, a plurality of devices may be stored in a relatively small amount of space until required for use.

Embodiments of the device intended for use in an operating room are preferably sterilizable. In accordance with an embodiment of the present invention, a device is sealed within a container, and the container subjected to sterilizing conditions so that the contents of the container, including the device, are sterilized. Such sterilizing conditions include, but are not limited to, exposure to gamma radiation or ethylene oxide. If desired, the device may be sterilized prior to being enclosed in the sealed container.

FIG. 15 illustrates a kit 100 for the controlled heating or cooling of a portion of the body in accordance with an embodiment of the present invention. Kit 100 includes a kit 94 comprising devices 82, 84, 86, 88, 90, and 92, as shown in FIG. 14, and additionally includes means for transferring heat to kit 94 which includes a system 104 of interconnecting inlet conduits 106, for providing a flow of water into the devices, a system 108 of interconnecting outlet conduits 10, for allowing water to flow out of the devices, and a control unit 102 for controlling the temperature of the water that flows into the devices. Control unit 102 is provided with means (not shown) for connecting to a source of water (not shown) and means (not shown) for connecting to a water disposal point (not shown). Control unit 102 includes a water reservoir (not shown) where water from the water source may be heated to a desired temperature. Control unit 102 thus controls the temperature of the water that flows through the waterproof chambers of kit 94, such that substantially the entire body of the patient is heated or cooled, as desired, by contact with the kit 94.

In kit 100 an inlet conduit is larger than an outlet conduit for each device. For example, an inlet conduit has a 1 cm diameter and an outlet conduit has a 1.2 cm diameter. In such a way, it is not possible to connect an inlet and outlet incorrectly.

In embodiments, in order to maintain the body of a patient at normal body temperature of 37° C., the temperature of the water flowing through kit 100 is approximately 45° C. The working pressure of the water flowing through the kit 100 should be less than 1 bar, and is preferably approximately 0.4-0.8 bar.

During use of kit 100 on a patient, it may become necessary to stop heating or cooling a portion of the body of the patient, such as, for example, the right arm. In this case, due to the modular construction of kit 94 and the fact that each device is heated or cooled separately, it is possible to cease the flow of water through the chamber of device 82. This may be accomplished by stopping the flow of water to the chamber of device 82, either by closing off inlet conduit 106 leading into device 82 or by removing inlet conduit 106 from device 82.

A particular feature of embodiments of the present invention is transparency to radiation, such as used in medical imaging modalities such as X-ray, radiation-emission and MRI. Thus, when the device is utilized to heat or cool a portion of the body of a patient, various diagnostic tests may be performed on the patient without requiring the removal of the device.

An additional feature of the embodiments of the present invention is useful when the device is in use on a patient, in the event that it becomes necessary to have access to specific parts of the patient, such as to administer an injection. A relatively small portion of the device may be bent away from the patient, so as to expose a limited portion of the body, without removing the entire device. In this manner, the device may continue to heat or cool the body of the patient.

It may be noted that, while kit 100 may be utilized on a patient in a hospital, embodiments of a device 94 may also be employed in an ambulance or helicopter or other emergency or mobile situation, where the fluid chambers of device 94 may be heated by electrical or chemical heating elements which will be discussed in detail below. The fluid chambers of device 94 are waterproof, as noted above with reference to valve 70 (FIGS. 7-10), such that the fluid contained within the chambers may be continually heated by heating elements while the device is being utilized in the mobile situation. Upon arrival at an appropriately equipped location, for example at a hospital, the source of electricity for the heating elements may be disconnected, and the patient may then be transferred, while still wearing the device 94 to the hospital. Due to the insulating effect of the device and especially when filled with a high heat-capacity fluid, the patient is relatively unaffected by the outside temperature. Once inside the hospital, the remaining components of the kit 100, i.e., system 104 of inlet conduits 106, system 108 of outlet conduits 110 and control unit 102, may be connected to device 94 so as to provide device 94 with a continuous flow of fluid for heating the patient and resuming active heating

With reference to FIG. 16 there is shown a kit 120 for the controlled heating or cooling of the upper surface 124 of a bed 122, in accordance with a further embodiment of the present invention. Kit 120 includes a plurality of rectangular devices 104, each similar in function to that of device 30 (FIGS. 3-4), a plurality 126 of interconnecting inlet conduits 128, a plurality 130 of interconnecting outlet conduits 132, and a control unit 102 for controlling the temperature of the water that flows into the waterproof chambers of devices 104. The operation and function of kit 120 are similar to those of kit 100 (FIG. 15), such that substantially the entire upper surface 124 of bed 122 is heated or cooled, as desired, thus facilitating the heating or cooling of a patient who is laying on the bed.

FIGS. 17-20 illustrate devices for the controlled heating or cooling of portions of the body in accordance with further embodiments of the present invention. The devices 140, 150, 160, and 170 shown in FIGS. 17-20 are configured as a square, circle, rectangular, and triangle, respectively, and may be employed for the controlled heating or cooling of any portion of the body wherein a device of a particular shape may be preferred.

While the device of the present invention has been discussed above with reference to particular configurations suitable for the heating or cooling specific portions of the body, it should be understood that the present invention is not limited to these specific configurations. Rather, the device of the present invention may have any configuration desired, so as to facilitate the heating or cooling of any other portion of the body such as, for example, a head.

Further, embodiments of the device of the present invention may be utilized for the controlled heating or cooling of objects other than portions of the body. For example, if it desirable to treat a patient by infusing with an injectable fluid such as, for example, a saline solution, it may be preferable to maintain the saline bag at a particular temperature, such as normal body temperature. In this case, a device according to embodiments of the present invention having a suitable configuration such as, for example, a rectangle may be utilized to heat the saline bag prior to the start of the infusion. Heating of the saline bag employing the teachings of the present invention may continue, if desired, for the entire time the patient receives the infusion. In this manner, the saline solution which is introduced into the patient's bloodstream will be heated to approximately the patient's body temperature, or higher than the patient's body temperature (for example to treat hypothermia) or lower than the patient's body temperature (for example to treat fever) prior to the infusion.

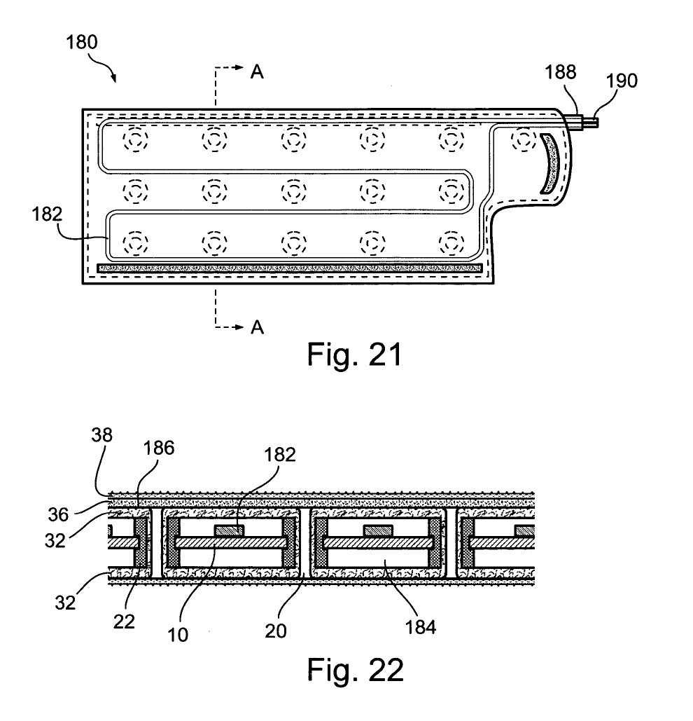

With reference to FIGS. 21 and 22, there is shown a device 180 for the controlled heating of a portion of the body in accordance with a further embodiment of the present invention. Device 180 includes a plastically deformable, substantially sheet element 10, which is similar in configuration and function to that discussed above with reference to FIG. 4. Device 180 is additionally provided with at least one heating element 182 which may be any suitable heating element, such as a component that produces heat from electricity, for example, due to ohmic resistance or such as a component that becomes hotter or cooler as a result of a chemical reaction, for example, a component including a sodium acetate solution (see for example U.S. Pat. No. 5,805,766). Heating element 182 has a configuration corresponding substantially to that of the plastically deformable element 10 and is positioned such that it is contact therewith. Heating element 182 defines a heat transfer component for device 180, and heating element 182 is shown as being located adjacent to plastically deformable element 10. If desired, heating element 182 may be attached to plastically deformable element 10 by any suitable means or may be formed integrally therewith.

In the embodiment shown, device 180 is provided with plastically deformable element 10, apertures 20, grommets 22, and latex layers 32 whose structures and functions are similar to those shown in FIG. 1. There is thus provided a thermo-insulating chamber 184 surrounding heating element 182 and plastically deformable element 10. While device 180 may be constructed by a method similar to that for device 30 (FIGS. 3-4), it should be understood that any other suitable method for creating a thermo-insulating chamber 184 for device 180 may be employed. The thermo-insulating chamber 184 defines a heat transfer component of device 180 and may be filled with any suitable fluid, such as air or water. The function of chamber 184 will be discussed in detail below. A surface 186 of latex layer 32 is preferably provided with a layer 36 of polyurethane elastomer sponge and a further layer 38 of terry cloth. The functions of layers 36 and 38 are similar to those discussed above with reference to FIG. 4.

The portion of the latex material at the portion 190 of the electrical heating element which extends beyond the periphery of the plastically deformable element 10 has been removed to expose an electrical connector 188. Electrical connector 188 may be connected to a control unit (not shown) and to a source of electricity (not shown) such that heating element 182 may be heated within thermo-insulating chamber 184.

When in use, heating element 182 is heated, thereby heating the fluid within thermo-insulating chamber 184 which defines the heat transfer component of the device 180. As the contents of chamber 184 are heated, the heat is transmitted via latex layer 32, polyurethane elastomer sponge layer 36, and terry cloth layer 38, such that a portion of the body in contact with layer 38 is heated.

While device 180 enables the controlled heating of a portion of the body, it is disadvantageous in that, in the event of a power outage, the thermo-insulating chamber 184 will quickly lose heat.

If desired, in addition to the provision of heating element 182, electrical connector 188, and thermo-insulative chamber 184, device 180 may be provided with additional means for heating and cooling of a portion of the body. This additional means may be similar to that of device 30 (FIGS. 3-4), such that chamber 184 would additionally act as a fluid chamber similar to fluid chamber 40 and would require a fluid inlet and a fluid outlet to be formed therefore.

In summary, embodiments of the present invention include a heat-generating element such as an electrical heating element to generate heat. Such embodiments may have a disadvantage of too quick heating, difficulty with exact regulation of the temperature and quick cooling when disconnected from a power source. In embodiments, the heat generating element are functionally associated with a component having a high-heat capacity, for example a chamber filled with a fluid such as water or an aqueous solution. Initially the heat-generating elements heat the fluid to a desired temperature, a relatively easy task to perform accurately due to the high heat capacity of water. Once the fluid is heated, disconnection from a power source leads only to a gradual and relatively slow reduction of temperature of the device, for example during transport from one location to another. In embodiments, the chamber is also provided with fluid inlets and outlets as described above. The utility of such a device is great, for example, a person is wrapped with such devices and the devices connected to an electrical power source of a helicopter to heat a fluid in the devices to heat the patient. When the helicopter lands at a hospital, the devices are disconnected from the power source of the helicopter but the devices maintain the body temperature of the patient. In the hospital the devices are attached to a heating fluid source.

In order to better understand embodiments of the present invention, as illustrated in FIGS. 1-4, reference is made to a method for producing the device having a fluid chamber, as discussed above.

Depending on the exact configuration of the device desired, a plastically deformable, substantially sheet element is chosen having a particular shape and size. While the plastically deformable element may be constructed of any flexible material, it is preferable to use an inexpensive metal foil such as aluminum. It will be appreciated by persons skilled in the art upon perusal of the description that, if desired, any other suitable metal such as, for example, gold, or any other flexible material may be used.

The edges of the plastically deformable element are folded over, such that there are no sharp edges which might tear through the latex which is to coat the plastically deformable element. A pair of aluminum tabs is provided on opposite ends of the plastically deformable element, at the locations where the inlet and outlet of the waterproof chamber are to be located. A plurality of apertures is provided throughout the plastically deformable element and a rigid grommet constructed of, for example, rubber, is provided in each of the apertures.

The plastically deformable element is dipped in a latex bath for a time sufficient to entirely coat the plastically deformable element with latex material. The duration of the latex bath depends on the desired thickness of the resulting latex layers formed on the plastically deformable element. Preferably, each latex layer should be approximately 0.1 mm thick. Typical latex layer thickness of 0.5, 0.8, or 1.0 mm may be desired. While latex is the preferred material, as it is inexpensive and is widely used in the field of medicine, it should be understood that, if desired, any suitable flexible material such as various other polymers may be used to coat the plastically deformable element. For example silicon rubber or neoprene rubber may be used. A particular feature of embodiments of the present invention is that the device may be formed of puncture resistant latex, which is important when the device may be in the vicinity of sharp objects surgical instruments. Additionally, it is important to note that embodiments of the device of the present invention are especially strong and, due to a novel construction, simple to employ on any patient, regardless of his medical condition or weight.

The latex material coating the plastically deformable element is cured, thus forming a deformable waterproof layer on either side of the plastically deformable element. It may be noted that a particular property of the latex material is that it will not stick to aluminum and that the deformable waterproof layers are sealed together at the edges of the plastically deformable element and through the apertures in the plastically deformable element. Thus, there is defined a fluid chamber around the plastically deformable element, the fluid chamber having substantially no weak points such as, for example, seams or welds. The fluid chamber is configured to correspond substantially to the plastically deformable element. In order to ensure sterility of the device, it may be sterilized by any suitable method such as, for example, being exposed to gamma radiation, as is known in the art.

If desired, the fluid chamber may be produced by dipping the plastically deformable element in a plurality of latex baths, each latex bath containing different latex additives chosen to impart specific properties to the latex such as, for example, increased elasticity, greater adhesion, increased resistance to puncturing, or any combination thereof. In this manner, specific properties, such as those noted above, may be imparted to the device of the present invention.

The presence of the grommets in the device prevents the latex coating the plastically deformable element from being punctured by the sharp edges of the apertures. The apertures prevent the aluminum foil from buckling or bending improperly. Furthermore, the presence of the grommets provides the device with a spacer component that prevents the latex around the aluminum element from ballooning when the plastically deformable element is bent, such as when in use on a portion of the body. The exact number and location of the apertures and grommets in the plastically deformable element is important in determining the size of the waterproof chamber and the thickness of the device. For example, if more apertures and grommets are disposed in the plastically deformable element, the device will be thinner.

It should be understood that, while in embodiments the present invention employs a foil layer as a plastically deformable element whose edges have been folded along the periphery of the foil layer (FIG. 2) and apertures in which there are disposed grommets, any other suitable means for blunting the edges of the foil layer and the edges of the apertures may be employed in order to prevent the latex coating the plastically deformable element from being punctured by the sharp edges of the foil layer and the sharp edges of the apertures.

After the fluid chamber has been formed, a portion of the latex material coating each of the aluminum tabs on the ends of the plastically deformable element is cut off, thus providing the fluid chamber with a pair of openings defining an inlet and an outlet. The inlet and outlet are each provided with a self-sealing valve, such that fluid may flow into the fluid chamber only via the inlet and fluid may flow out of the fluid chamber only via the outlet.

If desired, the device may be provided with an outer thermo-insulating layer, on the latex layer that is further away from the body portion. The purpose of this outer thermo-insulating layer is that, when the device is in use, for example for heating a body portion, and the fluid chamber is filled with fluid, less heat lost to the surroundings and thus the temperature of the fluid within the chamber may be more easily maintained.

If desired, the device may be provided with an outer thermo-insulating layer, on the latex layer that is closer to the body portion. The purpose of this outer thermo-insulating layer is that, when the device is in use, for example for heating a body portion, heat from the chamber will be transmitted to the body portion more slowly and evenly.

A suitable material for use as a thermo-insulating layer for either of the above purposes is a polyurethane or elastomer sponge, although any suitable material may be employed.

While the outer surface of the device is constructed of a soft material that is pleasant to the touch, there may be a need to cover the surface of the device with another outer material if, for example, a patient is allergic to latex. Thus, if desired, the device may be provided with at least one outer hypoallergenic insulating layer of any suitable material such that, when in use on a portion of the body, a hypoallergenic insulating layer will be in contact with the body portion. In the event that the patient on whom the device is being used is allergic or otherwise sensitive to latex, the provision of an outer insulating layer will prevent any adverse reaction to contact of the skin with the latex. A material suitable for use as an outer hypoallergenic insulating layer on the latex of the device may be, for example, terry cloth. Such a layer has a further advantage of wicking away sweat from the body.

Embodiments of devices of the present invention are provided with a mechanism for securing allowing maintenance of the devices in position about a portion of the body. The exact mechanism for securing may be any suitable securing means such as adhesive strips or “hook and loop” fasteners, as discussed above. Alternatively, any suitable securing means may be employed.

In order to simultaneously produce a plurality of devices in accordance with the teachings of the present invention, a plurality of plastically deformable, substantially sheet elements having different or similar shapes may be fastened to a rod, such that they descend therefrom. Thus, all of the elements may be lowered simultaneously into a latex bath and dipped thereinto. Such a mass-production method reduces production costs.

In order to better understand embodiments of the present invention as illustrated in FIGS. 21-22, reference is made to a method for producing the device having an electrical heating element, as discussed above. The method is similar to that discussed above, with reference to FIGS. 1-4, for producing the device having a fluid chamber, with the additional steps of: providing the plastically deformable element with an electrical heating element formed integrally therewith; providing the electrical heating element with an electrical connector disposed at the end thereof such that the electrical connector extends beyond the periphery of the plastically deformable element; and removing a portion of the latex material at the end of the electrical heating element which extends beyond the periphery of the plastically deformable element to thereby expose the electrical connector.

Reference is now made to a method for producing a self-sealing valve, as illustrated in FIGS. 7-10. According to the method, there is provided a planar former (preferably generally rectangular) having a size corresponding to the size of the valve to be produced. The former is dipped in a polymer (e.g., a suitable polymer such as materials suitable from which to fashion a device of the present invention as discussed above) bath for a time sufficient to entirely coat the former with uncured polymer. Preferably, the thickness of the polymer coating the substrate is approximately 0.4-0.5 mm thick. The polymer material coating the former is cured, thus forming first and second deformable waterproof layers on either side of the former, the first and second deformable waterproof layers being sealed together at the edges thereof, such that the first and second deformable waterproof layers define a cavity having a cross-sectional configuration corresponding to the substrate.

A portion of the polymer material coating the former is cut off at each end thereof, so as to provide the cavity with a pair of openings defining an inlet and an outlet, the remaining polymer material defining a passageway from the inlet to the outlet. Thereafter, the former is removed from the passageway.

Due to the relatively simple construction of embodiments of a device of the present invention, the materials used are inexpensive and the method by which the device may be produced is inexpensive as well. Therefore, after use, embodiments of a device of the present invention may be discarded. This is an advantage over more expensive prior art devices which are not disposable and have to be sterilized before each use.

Embodiments of the device of the present invention are configured to be reusable that is to be cleaned and/or washed and/or sterilized after a use and to thus recycle the device for further use.

Although described with respect to treating the heating and cooling of a portion of the body, the teachings of the present invention are applicable to many different applications. Specific applications include but are not limited to the heating and cooling of a portion of the body of a patient during a surgical procedure or while a patient is lying in a hospital bed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described with reference to specific embodiments thereof, it is evident that many alternatives, modifications and variations will be apparent to those skilled in the art.

Accordingly, the present invention is intended to embrace all such alternatives, modifications, and variations that fall within the spirit and broad scope of the appended claims.

All publications, patents, and patent applications mentioned in this specification are herein incorporated in their entirety by reference, to the same extent as if each individual publication, patent, or patent application was specifically and individually indicated to be incorporated herein by reference. The above notwithstanding, citation or identification of any reference in this application shall not be construed as an admission that such reference is available as prior art to the present invention.

What is claimed is:

1. A device for the controlled heating or cooling of a portion of the body, comprising:
a contact surface;
a plastically deformable, substantially sheet element configured to define the shape of said contact surface; and
a heat transfer component, associated with said plastically deformable element, for adjusting the temperature of said contact surface.

2. The device according to claim 1, said contact surface configured to make contact with a portion of the body such that either heat is transferred from the body portion to said contact surface or heat from the body is transferred to said contact surface.

3. The device according to claim 1, said heat transfer component comprising a chamber having walls of a flexible material.

4. The device according to claim 3, said chamber functionally associated with said deformable sheet element such that said deformable sheet element substantially defines the shape of said chamber.

5. The device according to claim 4, wherein said deformable sheet element is found substantially within said chamber.

6. The device according to claim 4, wherein said deformable sheet element is found substantially outside said chamber.

7. The device according to claim 3, wherein said walls of said chamber are substantially waterproof.

8. The device according to claim 7, wherein the size and shape of said chamber are substantially similar to the size and shape of said plastically deformable element.

9. The device according to claim 3, said material being elastic.

10. The device of claim 3, wherein one of said walls of said chamber defines said contact surface.

11. The device of claim 7, further comprising an external layer defining said contact surface, said external layer in intimate contact with at least one of said walls of said chamber.

12. The device of claim 11, wherein said external layer is in direct intimate contact with said at least one wall.

13. The device of claim 11, wherein between said external layer and said at least one wall there is at least one further layer, so that said external layer and said at least one wall are in indirect intimate contact.

14. The device according to claim 7, said chamber configured to hold water.

15. The device according to claim 3, said chamber provided with a fluid inlet configured to couple with a fluid source.

16. The device according to claim 15, wherein said fluid inlet comprises a quick-release fitting configured to couple with the fluid source

17. The device according to claim 15, wherein said fluid inlet comprises a one-way valve configured to prevent fluid flow from said inlet if uncoupled from the fluid source.

18. The device according to claim 17, wherein said one-way valve of said fluid inlet comprises a self-sealing valve.

19. The device according to claim 3, said chamber provided with a fluid outlet configured to couple with a fluid drain.

20. The device according to claim 19, wherein said fluid outlet comprises a quick-release fitting configured to couple with the fluid drain.

21. The device according to claim 19, wherein said fluid outlet comprises a one-way valve configured to prevent fluid flow from said outlet if uncoupled from the fluid drain.

22. The device according to claim 21, wherein said one-way valve of said fluid outlet comprises a self-sealing valve.

23. The device according to claim 1, said plastically deformable element comprising a metal.

24. The device according to claim 3, said heat transfer component being flexible.

25. The device according to claim 24, said heat transfer component being elastic.

26. The device according to claim 24, said heat transfer comprising a material selected from the group consisting of elastomer, rubber, latex, neoprene rubber and silicon rubber.

27. The device according to claim 3, further including a spacer component for maintaining opposing walls of said chamber in a substantially defined spaced apart relation relative to each other.

28. The device according to claim 27, wherein said spacer component comprises a plurality of substantially rigid elements, each of said substantially rigid elements being retained within a portion of said plastically deformable element.

29. The device according to claim 1, said heat transfer component comprising at least one heating element, said at least one heating element having a shape corresponding substantially to that of said plastically deformable element.

30. The device according to claim 29, said at least one heating element configured to generate heat.

31. The device according to claim 29, said at least one heating element being intimately associated with said plastically deformable element.

32. The device according to claim 29, said at least one heating element being integrally formed with said plastically deformable element.

33. The device according to claim 29, wherein at least one said heating element is an electrical heating element.

34. The device according to claim 29, wherein said at least one heating element is a chemical heating element.

35. The device according to claim 1, said heat transfer component comprising:
a fluid chamber, said fluid chamber functionally associated with said deformable sheet element such that said deformable sheet element substantially defines the shape of said fluid chamber; and
at least one heating element, said at least one heating element having a shape corresponding substantially to that of said plastically deformable element.

36. The device according to claim 1, wherein said plastically deformable element is substantially a sheet having a shape, said shape selected from the group consisting of square, circular, oval, rectangular, comb-shaped, star shaped and triangular.

37. The device according to claim 1, wherein said plastically deformable element is configured to substantially wrap about a portion of a body.

38. The device according to claim 37, further including a mechanism for securing the device about a portion of a body.

39. The device according to claim 38, the device being substantially of planar topology with first and second edges, said securing mechanism including a first component proximal to said first edge, whereby, when the device is positioned about a portion of a body, said first component of said securing mechanism is brought into registration with said second edge portion, and said first securing component is actuated so as to maintain the device in position about the portion of the body.

40. The device according to claim 39, said securing mechanism including a second component located proximal to said second edge whereby, when the device is positioned about a portion of a body and said first component of said securing mechanism is brought into registration with said second component, said securing mechanism is actuated so as to maintain the device in position relative to the portion of the body.

41. The device according to claim 1, enclosed within a sealed container.

42. The device according to claim 41, wherein the device is sterilized prior to being enclosed in said sealed container.

43. The device of claim 41, wherein said container is sterilizable.

44. The device according to claim 1, being substantially transparent to at least one imaging modality selected from the group consisting of X-ray imaging, radiation-emission imaging and magnetic resonance imaging.

45. A method for heating a vessel of injectable fluid, the method comprising wrapping a vessel of injectable fluid with a device of claim 1 and activating said heating element so as to heat the injectable fluid.

46. A kit for the controlled heating or cooling of a portion of a body, comprising:
at least two devices for the controlled heating or cooling of a portion of the body, each said device including:
a contact surface;
a plastically deformable, substantially sheet element configured to define the shape of said contact surface; and
a heat transfer component, associated with said plastically deformable element, for adjusting the temperature of said contact surface.

47. The kit of claim 46, further comprising a component for exchanging energy with said heat transfer components of said devices.

48. The kit of claim 47, said component for exchanging energy comprising:
at least one inlet conduit associated with each of said devices, for providing a flow of fluid into said devices;
at least one outlet conduit associated with each of said devices, for accepting a flow of fluid out of said devices; and
a control unit for controlling the temperature of said heat transfer components.

49. The kit of claim 48, said inlet conduits connecting said heat transfer components to a fluid source and said plurality of outlet conduits connecting said heat transfer components to a fluid disposal point, said control unit controlling the temperature of fluid flowing via said plurality of inlet conduits into said heat transfer components, thereby controlling the temperature of said heat transfer components.

50. A kit according to claim 47, said heat transfer component of each said device comprising at least one electrical heating element further comprising:
a controllable electrical power source for providing electricity to said electrical heating elements; and
a control unit for controlling the amount of power provided by said power source to said electrical heating elements, thereby controlling the temperature of said heat transfer components.

51. The kit of claim 50, wherein a shape of a said electrical heating element substantially corresponds to that of a respective said plastically deformable element.

52. The kit according to claim 50, wherein a said electrical heating element is integrally formed with a said plastically deformable element of a respective said device.

53. The kit according to claim 47, said heat transfer component of a said device comprising at least one chemical heating element.

54. The kit according to claim 53, wherein said at least one chemical heating element includes a controllable chemical source for initiating a chemical reaction to change the temperature of a respective said heat transfer component.

55. A self-sealing valve fabricated of a flexible material, said valve defined by a cylindrical portion of said flexible material, the valve having an inner surface and an outer surface, said inner surface defining a passageway through the valve, said passageway having an inlet end and an outlet end, the valve characterized in that, fluid flowing through a tubular element inserted into said passageway at said inlet end travels from said inlet end towards said outlet end and when no fluid flows through the tubular element towards said inlet, a pressure differential between a fluid on said outer surface at said outlet end and a fluid inside said passageway forces said passageway to be closed, thereby preventing a flow of fluid from said outlet end towards said inlet end.

56. A method for heating or cooling at least a portion of the body of a person, comprising the steps of:
(a) providing a device having:
a contact surface;
a plastically deformable, substantially sheet element configured to define the shape of said contact surface; and
a heat transfer component, associated with said plastically deformable element, for adjusting the temperature of said contact surface;
(b) positioning said device relative to a portion of a body of a person and manipulating said plastically deformable element such that said contact surface of said device is substantially in contact with said portion of the body; and
(c) exchanging energy with said heat transfer component of said device so as to change the temperature of said heat transfer component, thereby heating or cooling at least a portion of the body.

57. The method of claim 56, wherein said exchanging energy includes providing electrical power to an electrical heating element functionally associated with said heat transfer component.

58. The method of claim 57, wherein said device further comprises a chamber having walls of a flexible material including a fluid, and said electrical heating element heats said fluid.

59. The method of claim 56, wherein said device further comprises a chamber having walls of a flexible material, a fluid inlet and a fluid outlet; and wherein said exchanging energy includes providing a flow of fluid through said chamber in said inlet and out said outlet.

60. The method of claim 59, wherein said fluid is at a temperature higher than a portion of the body so as to affect heating of the portion.

61. The method of claim 59, wherein said fluid is at a temperature lower than a respective portion of the body so as to effect cooling of the portion.

62. The method of claim 56, wherein said exchanging energy includes initiating a chemical reaction that changes the temperature of a said heat transfer component.

63. The method of claim 62, wherein said chemical reaction is exothermic.

64. The method of claim 62, wherein said chemical reaction is endothermic.

65. A method according to claim 56, said device being substantially of planar topology with a first and second edge portion, said step (b) including plastically deforming said device around the body portion until said edge portions are proximally disposed.

66. The method of claim 65, wherein between said first and second edge portions there is a gap allowing access to a part of a surface of the body portion.

67. The method of claim 65, wherein said first and second edge portions substantially abut so as to substantially wrap the body portion with said device.

68. The method of claim 65, wherein said first and second edge portions substantially overlap as to wrap the body portion with said device.

69. A method according to claim 68, said method including the additional step, in between steps (b) and (c), of providing a mechanism for securing said device to the portion of the body.

70. The method according to claim 69, said device further including a mechanism for securing the device about a portion of a body.

71. The method according to claim 56, further comprising step (d) while said device is positioned relative to the portion of the body, imaging the portion using a medical imaging modality selected from the group consisting of X-ray imaging, radiation emission imaging and magnetic resonance imaging.

72. A method for manufacturing a device for the controlled heating or cooling of a portion of a body, the method comprising the steps of:
(a) providing a plastically deformable, substantially sheet element including a plurality of apertures;
(b) dipping said plastically deformable element in a bath of uncured polymer to coat said plastically deformable element with a polymer film; and
(c) curing said uncured polymer so as to form a flexible, walled chamber in which said plastically deformable element is contained.

73. The method of claim 72, further comprising, in between steps (a) and (b):
(d) dipping said plastically deformable element in at least one additional bath of uncured polymer to coat said plastically deformable element with at least one additional polymer film.

74. The method of claim 73, wherein said at least one additional bath of uncured polymer in step (d) and said bath of uncured polymer in step (b) contain different components.

75. The method of claim 72, further comprising, in between steps (a) and (b):
(d) blunting the outer edges of said plastically deformable element.

76. The method of claim 75, wherein said blunting of edges comprises folding over said edges of said plastically deformable element.

77. The method of claim 72, further comprising, in between steps (a) and (b):
(d) blunting the edges of said apertures.

78. The method of claim 77, wherein said blunting of aperture edges comprises placing a grommet in an aperture.

79. The method of claim 72, wherein said plastically deformable element includes a first tab and wherein, during said dipping, said plastically deformable element is held at said first tab by a holder.

80. The method of claim 79, wherein said holder comprises a fluid inlet and further comprising:
(e) subsequent to (c), forcing fluid through said fluid inlet into said chamber thereby determining the integrity of walls of said chamber.

81. The method of claim 72, said plastically deformable element comprising an electrical heating element.

82. The method of claim 81, wherein said plastically deformable element includes a first tab, wherein during said dipping, said plastically deformable element is held at said first tab by a holder and further comprising:
(f) detaching said first tab from said holder so as to define an opening into said chamber; and
(g) providing an electrical connector through said opening to allow provision of said electrical heating element with electrical power.

83. The method of claim 79, further comprising:
(f) detaching said first tab from said holder so as to define an opening into said chamber.

84. The method of claim 83, wherein said plastically deformable element includes a second tab, further comprising:
(h) subsequent to (c), cutting of a portion of polymer from said second tab so as to define an opening into said chamber.

85. The method of claim 84, further comprising placing connectors in said openings so as to define a fluid inlet into and a fluid outlet out of said chamber.

86. The method of claim 85, said connectors comprising unidirectional valves.

87. The method of claim 85, said connectors comprising self-sealing valves.

88. The method of claim 72, further comprising:
(i) subsequent to (c), sterilizing said chamber.

89. The method of claim 88, comprising, subsequent to (c), placing said chamber in a sterilizable packaging.

90. A method of producing a self-sealing valve, said method comprising the steps of:
(a) providing a substantially planar former;
(b) dipping said former in a bath of uncured polymer to coat said former with a polymer film;
(c) curing said uncured polymer so as to form a flattened, flexible-walled cylinder;
(d) removing said flattened cylinder from said former; and
(e) cutting a portion of said flattened cylinder to provide an aperture therein;
thereby forming a flexible walled flattened tube having at least two apertures that constitutes the self-sealing valve.

91. The method of claim 90, wherein subsequent to said dipping said polymer film entirely coats said former.

92. The method of claim 90, wherein subsequent to said dipping at least part of said former is bare of said polymer film.

93. The method of claim 90, wherein step (d) is performed prior to performing step (e).

94. The method of claim 90, wherein step (e) is performed subsequent to performing step (d).
